(19) United States
(12) Plant Patent Application Publication
(10) Pub. No.: US 2003/0135900 P1
(43) Pub. Date: Jul. 17, 2003

(54) Miniature rose variety POULset

(76) Inventors: L. Pernille Olesen, Fredensborg (DK); Mogens N. Olesen, Fredensborg (DK)

Correspondence Address: Poulsen Roser Pacific, Inc., 620 S. Front Street, Central Point, OR, 97502, US

(21) Appl. No.: 10/170,239

(22) Filed: Jun. 11, 2002

Related U.S. Application Data
(63) Continuation of application No. 09/611,215, filed on Jul. 6, 2000, now abandoned.

(51) Int. Cl. A01H 5/00

(52) U.S. Cl. PLT/121

(57) ABSTRACT

A new miniature rose plant which has abundant, non-fading, pink flowers and attractive foliage. The variety successfully propagates from softwood cuttings and is suitable for year round production in commercial glasshouses. This new and distinct variety has shown to be uniform and stable in the resulting generations from asexual propagation.

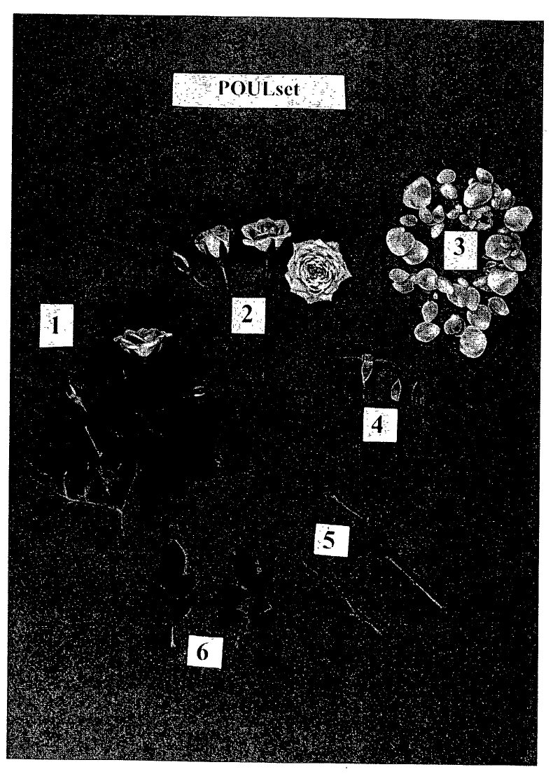

### SUMMARY OF THE INVENTION

The present invention constitutes a new and distinct variety of miniature rose plant which originated from a controlled crossing between an unnamed seedling and ‘POULsint’. The two parents were crossed and the resulting seeds were planted in a controlled environment. The new variety is named ‘POULset’.

The new rose may be distinguished from its seed parent, an unnamed seedling, by the following combination of characteristics:

1. The unnamed variety has taller grwoth than ‘POULset’.

2. The unnamed variety has larger flowers than ‘POULset’.

The new variety may be distinguished from its pollen parent, ‘POULsint’ created by the same inventors, by the following combination of characteristics:

1. The petals of ‘POULsint’ are orange-red, where those of ‘POULset’ are pink.

2. The growth of ‘POULsint’ is more vigorous than that of ‘POULset’.

3. The flowers of ‘POULsint’ grow in tops suitable for use as a spray cutflower rose.

The objective of the hybridization of this rose variety for commercial greenhouse culture was to create a new and distinct variety with unique qualities, such as:

1. Uniform and abundant flowers;

2. Moderate and compact growth;

3. Year-round flowering under glasshouse conditions;

4. Suitability for production from softwood cuttings in pots;

5. Durable flowers and foliage which make a variety suitable for distribution in the floral industry.

The combination of qualities of this variety represents significant improvement over previously available commercial cultivars of this type and distinguishes ‘POLJtset’ from all other varieties of which we are aware. As part of their rose development program, L. Pernille Olesen and Mogens N. Olesen germinated the seeds from the aforementioned hybridization and conducted evaluations on the resulting seedlings in a controlled environment in Fredensborg, Denmark.

‘POULset’ was selected by the inventors as a single plant from the progeny of the hybridization in Fredensborg, Denmark.. Asexual reproduction of ‘POULset’ by cuttings and traditional budding was first done by L. Pernille and Mogens N. Olesen in Fredensborg, Denmark. This initial and other subsequent propagations conducted in controlled environments have demonstrated that the characteristics of ‘POULset’ are true to type and are transmitted from one generation to the next.

### BRIEF DESCRIPTION OF THE DRAWING

The accompanying color illustration shows as true as is reasonably possible to obtain in color photographs of this type, the typical characteristics of the buds, flowers, leaves, stems, and a plant of ‘POULset’. Specifically illustrated in SHEET 1:

1. Stem or entire plant showing branching and the attachment of leaves, buds, and peduncles;

2. Flower bud, partially opened bud, and open bloom;

3. Flower petals, detached;

4. Sepals, receptacle, and pedicel;

5. Flowering stem as well as a bare stem exhibiting thorns;

6. Leaves.

### DETAILED DESCRIPTION OF THE VARIETY

The following is a description of ‘POULset’, as observed in its growth in glasshouses in Half Moon Bay, Calif., on plants aged 16 weeks. Color references are made using The Royal Horticultural Society (London, England) Colour Chart, 1995, except where common terms of color are used. For a comparison, several physical characteristics of the rose variety ‘POULfuna’, a miniature rose variety from the same inventors described and illustrated in U.S. Plant Pat. No. 10,628 and issued on Oct. 6, 1998 are compared to ‘POULset’ in Chart 1.

CHART 1

| | ’POULset’ | ’POULfuna’ |
|---|---|---|
| Flower color on | Red Group 52D. | Red-Purple Group 57C. |
| upper petal of | | |
| open bloom. | | |
| Flower color on | Red Group 49A. | Red-Purple Group 57D. |
| upper petal of | | |
| open bloom. | | |

Parents:

Classification:

Botanical.—Rosa hybrida.

Commercial.—

### FLOWER AND FLOWER BUD

Blooming habit:

Flower bud:

Size.—Upon opening, 19 to 20 mm in length from base of receptacle to end of bud.

Bud form.—Pointed Ovoid

Bud color.—As sepals unfold, Red Group 49B. Red Group 49B at ¼ opening.

Sepals.—Sepal length is 12 to 15 mm. Sepal width is 6 to 8 mm. Green Group 137A. Weak foliaceous appendages on three of the five sepals. Surfaces of sepals slightly pubescent. Stipitate glands are few, almost absent.

Receptacle.—Surface: Smooth, with small white hairs. Shape: Funnel. Size: Small; 5 mm (h) ×6 mm (w). Color: Yellow-Green Group 144A.

Peduncle.—Surface: Smooth Length: 7 to 9 mm average length. Color: Yellow-Green Group 144A. Strength: Upright.

Borne.—Singularly.

Flower bloom:

Fragrance.—Very light; floral.

Duration.—As a pot plant, flowers last from 8 to 12 days. As a cut flower, 3 to 4 days.// The blooms have a duration on the plant of approximately to days.// Petals fall cleanly away from plant.

Size.—Large for a 6 cm pot rose. Average flower diameter is 30 mm when open.

Form.—Shape of flower when viewed from the side: Upon opening, upper part: Flat. Upon opening, lower part: Convex. Open flower, upper part: Flat. Open flower, lower part: Flattened convex.

Petalage.—Very double; average range: 45 petals under normal conditions with 12 petaloids.

Color:

Upon opening, petals.—Outermost petals: Upper Surface: Red Group 48C. Reverse Side: Red Group 49A. Innermost petals: Upper Surface: Red Group 48C. Reverse Side: Red Group 49A.

Upon opening, basal petal spots.—Outermost petals: Outer Side: Orange-Yellow Group 1C. Inner Side: Orange-Yellow Group 1C. Innermost petals: Outer Side: Orange-Yellow Group 1C. Inner Side: Orange-Yellow Group 1C.

After opening, petals.—Outermost petals: Upper Surface: Red Group 52D. Reverse Side: Red Group 49A. Innermost petals: Upper Surface: Red Group 52D. Reverse Side: Red Group 49A.

After opening, basal petal spots.—Outermost petals: Outer Side: Yellow-Green Group 1C. Inner Side: Yellow-Green Group 1C. Inntermost petals: Outer Side: Yellow-Green Group 1C. Inner Side: Yellow-Green Group 1C.

General Tonality: On open flower Red Group 52D. No change in the general tonality at the end of the 3d day. Afterwards, general tonality is Red Group 49A.

Petals:

Petal Reflex.—Petals reflex somewhat.

Petal Margin.—Uniform.

Shape.—Round.

Petaloids.—Present; quantity, 9 to 12.

Thickness.—Average.

Arrangement.—Imbricated.

Reproductive Organs:

Pollen.—Color: Yellow Group 17B. Quantity: Scant.

Anthers.—Size: 1 mm long. Color: Yellow-Orange Group 216. Quantity: 30-35.

Filaments.—Color: Green Yellow Group 1B. Length: 3-4 mm.

Stigmas.—Slightly superior in location to anthers. Color: Yellow-Green Group 149D. Quantity: 20-25.

Styles.—Color: Yellow-Green Group 149D. Length: 4-5 mm.

### PLANT

Plant growth: Moderate, compact. When grown as a 6 cm pot plant, the average height of the plant itself is to 12 cm and the average width is to 17 cm. The average height of the plant itself is 18 cm and the average width is 25 cm.

Stems:

Color.—Young wood: Green Group 137C. Older wood: Green Group 137B.

Prickles.—Incidence: Moderate. Size: Average length: 2 to 3 mm. Color: Yellow-Green Group 145D. Shape: Linear.

Surface.—Young wood: Smooth. Older wood: Smooth. Length: Stem length is 30 cm on average. Internode length is 3 to 5 cm.

Plant foliage: Normal number of leaflets on normal leaves in nmddle of the stem: 5 leaflets.

Leaf size.—Small; 51 to 54 mm (l)×32 to 36 mm (w).

Ouantity.—Very abundant.

Color.—Upper Leaf Surface: Green Group 137A. Lower Leaf Surface: Green Group 138B. Juvenile foliage: Upper foliage, Green Group 138A. Lower foliage, Green Group 138B. Anthocyanin intonation: No.

Plant leaves and leaflets:

Stipules.—Size: 10 mm (l)×4 mm (w). Color: Green Group 143B. Presence of stipitate glands: None. Present are fine white hairs.

Petiole.—Length: 12 to 14 mm. Color: Green Group 143B. Underneath: Green Group 144A. No thorns or prickles. Margins: Green Group 137A.

Rachis.—Color: Green Group 143B. Underneath: Green Group 144A. No thorns or prickles. Margins: Green Group 137A.

Leaflet.—Edge: Serrated. Shape: Ovate. Texture: Matte Finish; thin.

Disease resistance: Average resistance to mildew, black spot, and Botrytis under normal growing conditions in Half Moon Bay, Calif.

Cold hardiness: ‘POULset’ has been found to be resistant to damage from cold, heat and drought in USDA Zone 7.

1. A new and distinct variety of rose plant of the miniature class, substantially as herein illustrated and described as a distinct and novel rose variety due to its abundant, pink flowers, moderate and compact growth, year round flowering under glasshouse conditions, suitability for production from softwood cuttings in pots, and durable flowers and foliage which make the variety suitable for distribution in the floral industry.
